(19) United States
(12) Patent Application Publication
(10) Pub. No.: US 2019/0151496 A1
(43) Pub. Date: May 23, 2019

(54) NOVEL ACTIVE INGREDIENT IN CICATRIZATION AND USE THEREOF

(71) Applicant: URGO RECHERCHE INNOVATION ET DEVELOPPEMENT, Chenove (FR)

(72) Inventor: Christelle LAURENSOU, Dijon (FR)

(21) Appl. No.: 16/254,211

(22) Filed: Jan. 22, 2019

Related U.S. Application Data
(63) Continuation of application No. 12/601,699, filed on Apr. 23, 2010, now abandoned.

(30) Foreign Application Priority Data
May 25, 2007 (FR) 0755271

(51) Int. Cl. A61L 15/44; A61K 8/81; A61K 31/7028; A61L 15/26; A61K 31/795; A61Q 19/00

(52) U.S. Cl. CPC A61L 15/44; A61K 8/8158; A61K 31/7028; A61L 15/26; A61L 2300/404; A61Q 19/00; A61L 2300/41; A61L 2300/412; A61L 2300/232; A61K 31/795

(57) ABSTRACT

The present invention relates to the use of a known compound as an agent for promoting and/or accelerating fibroblast proliferation and/or differentiation and, consequently, cicatrization. This compound is a copolymer of a 2-methyl-2-[(1-oxo-2-propenyl)amino]-1-propanesulphonic acid salt and of propenoic acid 2-hydroxyethyl ester. It may be used alone or in combination with other active substances for inducing or accelerating cicatrization. This compound, alone or in combination with another active substance, may be administered directly on the wound and the surrounding area or the mucous membranes, by topical application. It may also be used ex vivo, in particular for generating cells for skin grafts.

An object of the present invention is essentially a novel use of a compound which is a copolymer of a 2-methyl-2-[(1-oxo-2-propenyl)amino]-1-propanesulfonic acid salt and of 2-hydroxyethylpropenoate ester.

More specifically, the present invention relates to the use of this compound as an agent for promoting and/or accelerating fibroblast proliferation and/or differentiation, in particular for the preparation of a composition intended to promote healing, or alternatively for the preparation of a composition intended to be used ex vivo for generating cells for skin grafts.

The invention also relates to a dressing for the treatment of wounds containing this compound, alone or in combination with other active substances.

It is known that open wounds, which include burns, ulcers, neuropathic ulcers, venous or arterial ulcers, diabetic ulcers, bedsores, blisters, exudative wounds, superficial dermo-epidermal lesions, which may be chronic or acute, represent the vast majority of wounds.

One of the primary objectives in treating the wound, whatever the nature or origin thereof, is to close this wound.

Moreover, it is known that wound healing is a natural biological phenomenon, human and animal tissues being capable of repairing localized lesions by means of repair and regeneration processes which are specific thereto.

In general, wound healing occurs in three phases: the detersion phase, the granulation phase and the epithelialization phase, each of these phases being characterized by specific cellular activities which cause the repair process to progress according to precise chronological sequences.

The detersion phase is characterized by the appearance of early inflammatory phenomena. Immediately following the trauma, secretions originating from blood and lymph vessels begin. Coagulation is induced by activation of thrombokinase which is released with the correlative formation of fibrin. After approximately 10 minutes, exudation begins, which will provide defense against infection and detersion of the wound. Approximately 4 days later, the injury begins the proliferative phase. The organism begins to replace the loss of substance by a new tissue. For this purpose, the fibroblasts firstly produce mucopolysaccarides which will serve as a matrix for the development of the collagen fibers of the connective tissue. Between the 6th and 10th day, on average, the differentiation phase occurs. Collagen fibers maturation begins. The wound retracts under the influence of particular cells, myofibroblasts. The granulation tissue gradually becomes depleted of water and contains increasingly fewer vessels, thus becoming firmer. It is then converted into scar tissues which, in turn, will promote scar retraction.

It is over the course of the second healing phase, known as granulation phase, that the fibroblasts will play an important role. The granulation phase lasts approximately 3 weeks and, during the normal healing process, epithellalization is also under way. Fibroblasts multiply in the wound, as do keratinocyte precursor cells, from the edges of the wound, from the hair follicles and from the sweat glands. After 3 days, the fibroblasts produce collagen, the fibers of which become oriented according to the forces to which they are subjected. Their proliferation is governed by a certain number of factors. As a general rule, this proliferation is interrupted when the granulation tissue has replaced the loss of substance and when the fibroblast proliferation has risen to the level of the edges.

The fibroblast is one of the major agents in the healing process. In fact, around the 6th day, more than half the fibroblasts present in the wound transform into myofibroblasts. These cells are characterized by the presence, in their cytoplasm, of contractile myofibrils which cause contraction of the wound and, consequently, a decrease in the surface area of the wound and acceleration of the closing thereof.

These cells therefore have an essential role in wound contraction which is, a major phenomenon of spontaneous closing in cavity wounds. More than 40% of the myofibroblasts (containing a contractile protein, alpha smooth muscle actin) are present in the granulation tissue. When the wound is healed, these cells die, although the signal triggering their disappearance is not completely understood.

The fibroblast is also a key cell of the proliferative phase. The fibroblast will secrete collagen type III, and then subsequently collagen type I, and heparan sulfate, which are fundamental constituents of the dermal extracellular matrix, but also hyaluronic add, chondroitin sulfate, fibronectin and a collagenase, thereby resulting in the closing of the wound.

The healing process, even for small wounds or openings, can last for a period of a few hours, a few days or a few weeks, or even more, under certain circumstances, such as in the case of ulceration, where the wound may persist for much longer periods, I.e. months or years.

During this healing period, whether it lasts a short or long time, the wound may be subject to invasions of any type (pathogenic organisms or foreign substances) until the regeneration of a new tissue closes the opening completely.

In order to prevent any infection, the wound is normally treated in order to remove any contaminant (dust, debris, etc.) capable of introducing a pathogenic substance in the region of the damaged area. Debridement of the tissues in this area and disinfection are subsequently performed. In certain cases, a certain number of sutures are also inserted in order to facilitate wound closing. Once these steps have been carried out, the wound is kept in an environment favorable to healing. To do this, various types of dressings are used in order to prevent the intrusion of pathogens and/or proliferation thereof.

When treating a wound, it is therefore important to promote the wound-closing phenomenon in order to prevent, for example, invasion of the wound by microorganisms or foreign substances and, consequently, infection of the wound. As a result, it is necessary to have means for inducing or promoting the wound healing process.

In order to induce or accelerate wound healing, a certain number of active agents can be administered. These active agents may act at various levels and at the various phases of healing. They differ according to the stage and to the type of wound to be treated. These active agents may, for example, be fibroblast and/or keratinocyte growth factors (such as Basic Fibroblast Growth factor) or pseudo growth factors (such as mannose-6-phosphate), glycosaminoglycans (such as hyaluronic add, collagen, etc.), hormones (such as estradiol, DHEA, etc.), polysaccharides (such as dextran), etc.

It has been discovered, entirely surprisingly and unexpectedly, that it is also possible to promote and/or accelerate fibroblast proliferation and/or differentiation and, consequently, to promote healing by means of a compound which is a copolymer of a salt of 2-methyl-2-[(1-oxo-2-propenyl)amino]-1-propanesulfonic acid and of 2-hydroxyethylpropenoate ester.

Preferably, the abovementloned 2-methyl-2-[(1-oxo-2-propenyl)amino]-1-propanesulfonic acid salt is a sodium salt.

This copolymer is a product which is known as such in particular in the cosmetics field owing to its emulsifying-stabilizing properties and to its good thickening capacity.

Such a product is, for example, sold by the company SEPPIC under the trade name Sepinov EMT 10°.

Thus, according to a first aspect, an object of the present invention is the use of a copolymer of a salt of 2-methyl-2-[(1-oxo-2-propenyl)amino]-1-propanesulfonic acid and of 2-hydroxyethylpropenoate ester, for the preparation of a composition intended to promote and/or accelerate fibroblast proliferation in vivo or ex vivo, and in particular for the preparation of a composition intended to promote healing.

The abovementioned copolymer may be used in vivo or ex vivo.

As an example of an ex vivo application, mention may be made of the use of this compound for the preparation of a culture medium for promoting fibroblast proliferation. This use may prove to be useful for autologous dermal or dermo-epidermal sheet grafts. This is because, in this type of graft, cell culture techniques are used in order to obtain a sufficient surface area of skin from a small sample taken from the patient himself or herself. The use of the copolymer according to the present invention makes it possible to accelerate fibroblast proliferation and may therefore prove to be useful in this type of treatment.

In the context of its in vivo application, the abovementioned copolymer may be used for the preparation of a composition that will allow it to be applied directly on the wound and the surrounding area, or else on the mucous membranes, advantageously by topical application. Preferably, this copolymer is integrated into a dressing.

In general, the abovementioned copolymer may be used alone or in combination with other active substances which make it possible to induce or accelerate healing or which may have a favorable role In wound treatment, such as, for example, antimicrobial agents or pain relievers. treatment of the wound. Among the active substances that may be used in the context of the invention, mention may be made, by way of examples, of bactericidal or bacteriostatic agents (chloramine, chlorhexidine, silver salts, zinc salts, metronidazole, neomycin, etc.), agents for promoting healing (hormones, peptides, etc.), enzymes for promoting wound detersion (pepsin, trypsin, etc.), protease or metalloprotease inhibitors, pain relievers or local anesthetics (lidocaine, cinchocaine) or nonsteroidal anti-inflammatory drugs (ibuprofen, ketoprofen, fenoprofen, diclofenac).

Preferably, the abovementioned copolymer will be used in combination with a sulfated saccharide or a sulfated saccharide salt or complex. This sulfated saccharide may, for example, be a sucrose octasulfate in the form of a complex or of a salt with a metal such as Na, K, Ca, Mg, Ba, Al, Zn, Cu, Zr, Ti, Mn or Os, or with an organic base, such as an amino acid. Particularly advantageous results have been obtained when the abovementioned copolymer is used in combination with potassium sucrose octasulfate, which is an active ingredient known for its action in the healing process.

In vitro proliferation studies have made it possible to show, unexpectedly, that a copolymer of a 2-methyl-2-[(1-oxo-2-propenyl)amino]-1-propanesulfonic acid salt and of 2-hydroxyethylpropenoate ester exhibits an activity on fibroblast proliferation at a very low dose. Specifically, such an activity has been observed when the copolymer is present in the culture medium at a concentration of 0.00064 mg/mL.

It has also been shown that, particularly advantageously, the abovementioned copolymer also exhibits an activity over several days. Tests carried out in vivo have made it possible to detect an activity on fibroblast proliferation 72 hours after incorporation of said copolymer into the culture medium. This is particularly advantageous when the copolymer is incorporated into a dressing. Specifically, the same dressing may be used for several days while at the same time remaining active without the need for it to be changed.

In general, the copolymer used in the context of the present invention may be integrated into any type of composition, such as, in particular, a solution, a cream, a gel, a mass, in particular an elastomeric mass, or a dressing. The copolymer may be integrated into a compress. The term “compress” is intended to mean any type of absorbent material normally used in dressings, such as, for example, textile materials which may be absorbent nonwovens (based on viscose, for example) possibly combined with nonabsorbent nonwovens (such as polyester fibers). This may also involve superabsorbent fibers (such as the Lanseal® Fibres sold by Toyobo co, Ltd) or polyurethane foams.

In particular, this copolymer may be present in the mass constituting a dressing or on a separate layer of the dressing or alternatively in a coating covering the surface of the dressing, intended to come into contact with the wound in order to treat it.

The term “dressing” is herein intended to cover any type of known dressings, and preferably interface dressings. Such dressings are sold, for example, under the trade names Tulle Gras® (by Solvay Pharma), Physiotulle® (by Coloplast) or else Urgotul® (by Laboratoires Urgo) and described in patent EP 1 143 895.

These interface dressings are generally in the form of a mesh or of a netting coated with a mass, normally an elastomeric mass. They may also be constituted of a mass without a web or netting, having the form of a sheet which may or may not have through-holes, depending on the type of wound to which the dressing is applied (a sheet which has through-holes will preferably be used on an exudative wound when the mass has only a weak or no absorbent capacity, the holes thus allowing evacuation of the wound exudates).

The present invention also finds application for the preparation of dressings based on hydrogels or on hydrocolloids into which the abovementioned copolymer is incorporated. Known hydrocolloid-based dressings are, for example, sold under the names Algoplaque® (by Laboratoires Urgo), Duoderm® (by Convatec) and Comfeel® (by Coloplast). Such dressings are described in the following patent applications: FR 2 392 076, FR 2 495 473 and WO 98/10801 and EP 264 299.

The copolymer used in the context of the present invention may be incorporated into an absorbent element such as a compress or a foam, for example by depositing it on the surface intended to come into contact with the wound, as described in patent application WO 2006/007844.

The present invention also finds application for the preparation of interface dressings complexed with an absorbent layer such as a foam or a compress, or of a hydrocolloid complexed with an absorbent foam. Such dressings are known and are sold, for example, under the trade names UrgotulDuo® and Cellosorb® (by Laboratoires Urgo). In such dressings, the abovementioned copolymer may be incorporated into the mass and/or into the absorbent layer.

The interface dressings preferably used in the context of the present invention are constituted of an elastomeric mass, i.e. constituted from one or more elastomers chosen from polystyrene-olefin-styrene) block copolymers and one or more compounds chosen from plasticizers, tackifying resins and, if necessary, antioxidants.

Such elastomeric masses are well known to those skilled in the art and are described, for example, in “Advances in Pressure Sensitive Adhesive Technology” edited by Donatas Satas in April 1995 in chapter 7 “Wound dressings” pages 158 to 171.

When it is integrated into a dressing, such as an interface dressing, the abovementioned copolymer may be used at a concentration of from 1% to 5% by weight relative to the weight of the elastomeric mass. It has been shown that such a concentration is sufficient to activate fibroblast proliferation and, consequently, to promote healing.

In general, the abovementioned copolymer will be present in a dressing in an amount that may be between 0.1% and 20% by weight relative to the weight of the mass into which it is incorporated, and preferably between 1% and 5% by weight.

According to a second aspect, the present invention aims to cover the dressings described above which incorporate a copolymer of a salt of 2-methyl-2-[(1-oxo-2-propenyl)amino]-1-propanesulfonic acid and of 2-hydroxyethylpropenoate ester.

In order to demonstrate the activity of the abovementioned copolymer on fibroblasts, an in vitro study was carried out, using two types of experiments:
1. The copolymer alone or in combination with an active substance (in the present case, potassium sucrose octasulfate) was added directly to the fibroblast culture medium at various concentrations.

In this case, various dilutions of the copolymer, alone or in combination with the potassium sucrose octasulfate, were thus prepared and added to the culture medium.

Dilutions of the Sepinov EMT 10 copolymer at 0.011 mg/ml and of potassium sucrose octasuifate at 0.00064 mg/ml were thus tested in a culture medium comprising fibroblasts.
2. The copolymer alone or in combination with an active substance (in the present case, potassium sucrose octasulfate) was integrated into the matrix of an interface dressing, and the dressing was then applied to the culture wells containing the fibroblasts.

In this case, various interface dressings containing the copolymer at various concentrations, in combination or not in combination with potassium sucrose octasulfate, were produced according to the method described below. An interface dressing containing neither of these two active agents was also produced and tested, by way of comparison.

### Method for Producing the Tested Dressings

### a. Production of Elastomeric Masses

Various elastomeric masses (examples 1 to 5) were produced using the following constituents, In the proportions, by weight, mentioned in table 1:
Elastomer: block copolymer of poly(styrene-ethylene-butylene-styrene) (abbreviated to SEBS); Kraton G 1654 and G 1651 sold by the company Kraton;
Plasticizer: mineral oil: Ondina 917 sold by the company Shell;
Antioxidant: Irganox 1010 sold by the company Ciba Specialty Chemicals;
Fatty substance: petroleum jelly: Codex A petroleum jelly sold by Aiglon;
Hydrocolloid: sodium carboxymethylcellulose: CMC Blanose 7H4XF sold by the company Hercules;
Additional active substance: potassium sucrose octasulfate: sold by the company Euticals;
Copolymer of a 2-methyl-2-[(1-oxo-2-propenyl)amino]-1-propanesulfonic acid salt and of propenoic acid 2-hydroxyethyl ester: Sepinov EMT 10 sold by the company SEPPIC.

The elastomer-based mass was prepared by mixing in a Z-blade mixer at a setpoint temperature of 155° C.:
1. The styrene-ethylenebutylene-styrene triblock elastomers are mixed with half the mineral oil and with the antioxidant.
2. At 30 minutes, the petroleum jelly is added to the mixture.
3. At 40 minutes, the rest of the mineral oil is added.
4. At 55 minutes, the sodium carboxymethylcellulose, where appropriate the active agent(s) are added to the mixture.

The mixer is emptied at 70 minutes.

### b. Production of Dressings

Interface dressings constituted of a mesh coated with an elastomeric mass were produced using the abovementioned elastomeric masses.

More specifically, a mesh formed from a thermoset marquisette made of polyester (polyethylene terephthalate) yarns of 33 decitex in the warp and weft directions, having square mesh cells with an aperture of approximately 0.8 to 1 mm² (mesh 555 sold by the company MDB Texinov) was used here.

This mesh was coated with a layer of molten elastomeric mass at 135-145° C., and then the excess was removed by passing between two fixed rollers having a gap of 200 μm therebetween. The strip thus obtained was cut and then complexed with a protective polyester film having a thickness of 23 μm, on each of its sides, thus forming individual dressings packaged in impermeable pouches and sterilized under β-radiation at 25 kGy.

TABLE 1

| Constituents | Example 1 | Example 2 | Example 3 | Example 4 | Example 5 |
|---|---|---|---|---|---|
| Mineral oil (Ondina 917) | 75 | 74 | 70 | 62.38 | 69.9 |
| S-EB-S (Kraton G 1651) | 4.9 | 4.9 | 4.9 | | |
| S-EB-S (Kraton G 1654) | | | | 6 | 6 |
| Antioxidant (Irganox 1010) | 0.1 | 0.1 | 0.1 | 0.12 | 0.1 |
| Petroleum jelly (Codex A | 5 | 5 | 5 | 5 | 5 |
| petroleum jelly) | | | | | |
| Carboxymethylcellulose | 15 | 15 | 15 | 14 | 14 |
| (CMC Blanose 7H4XF) | | | | | |
| Sepinov EMT 10 | | 1 | 5 | 5 | 5 |
| Potassium sucrose | | | | 7.5 | |
| octasulfate | | | | | |

The effect of the copolymer of salt of 2-methyl-2-[(1-oxo-2-propenyl)amino]-1-propanesulfonic acid and of 2-hydroxyethylpropenoate ester on fibroblast proliferation was determined according to the following protocol.

### Demonstration of the Activity of the Abovementioned Copolymer on Fibroblast Proliferation

### Cells Used:

Type: pool of normal human dermal fibroblasts (NHDF) R9PF2

Culture: 37° C., 5% CO₂

Culture medium:
DMEM (Dulbecco's Modified Eagle Medium, Invitrogen 21969035)
2 mM L-glutamine (Invitrogen 25030024)
50 IU/mL penicillin (Invitrogen 15070063)
10% fetal calf serum (v/v, Invitrogen 10270098).

### Products Tested:

Dressings having the size of the wells, prepared as described previously using the elastomeric masses of examples 1 to 5, and also dilutions of the Sepinov EMT 10 copolymer to 0.011 mg/mL and of potassium sucrose octasulfate to 0.00064 mg/mL, were tested.

### Effects on Proliferation:

The fibroblasts were seeded into 12-well plates at low density (60% confluence), and then the cells were treated with the dilutions or pieces of dressings were applied to these plates and kept in place by means of a plug (called “test”). A control without dressing, without dilution but with plug was carried out (called “control”).

The cells were then incubated for 24 hours, 48 hours and 72 hours.

For each incubation time, tritiated thymidlne ([methyl-3H]-thymidine, Amersham TRK 686 2.5 μCl/mL final concentration) was added for the final 24 hours of incubation and then the DNA of the cells of the cell layers was extracted and purified and the radioactivity incorporated into the DNA was counted using a scintillation counter.

The results obtained are expressed as counts per minute (cpm), then as percentage relative to the control according to the following formula:

%_{control}=(cpmₜₑₛₜ/cpm_{control})×100

in which:

cpmₜₑₛₜ: number of counts per minute obtained with the test

cpm_{control}: number of counts per minute obtained with the control.

All experiments were carried out in triplicate.

The results obtained are given in tables 2 to 4 below.

TABLE 2
Results of the study of NHDF proliferation after incorporation of
tritiated thymidine at 24 hours

| Treatment | %_{control} |
|---|---|
| Control | 100 |
| Dressing according to example 1 | 130 |
| Dressing according to example 2 | 196 |

TABLE 3
Results of the study of NHDF proliferation after incorporation of
tritiated thymidine at 48 hours

| Treatment | %_{control} |
|---|---|
| Control | 100 |
| Dressing of example 1 | 181 |
| Dressing of example 2 | 260 |
| Dressing of example 3 | 506 |
| Dressing of example 4 | 456 |
| Dressing of example 5 | 243 |
| Potassium sucrose octasulfate at 0.011 mg/mL | 106 |
| Sepinov EMT 10 at 0.00064 mg/mL | 125 |
| Potassium sucrose octasulfate at 0.011 mg/mL and | 152 |
| Sepinov EMT 10 at 0.00064 mg/mL | |

TABLE 4
Results of the study of NHDF proliferation after incorporation of
tritiated thymidine at 72 hours

| Treatment | %_{control} |
|---|---|
| Control | 100 |
| Dressing according to example 1 | 150 |
| Dressing according to example 2 | 120 |
| Dressing according to example 3 | 493 |
| Dressing according to example 4 | 292 |
| Sepinov EMT 10 at 0.00064 mg/mL | 147 |

These in vitro experiments thus made it possible to demonstrate the role of the Sepinov EMT 10 copolymer on fibroblast proliferation and also the combined activity of this copolymer and of potassium sucrose octasulfate on these same cells.

The effect obtained is particularly advantageous when the Sepinov EMT 10 copolymer is integrated into an elastomeric mass at a concentration of 5% of the total mass, this being after 48 h and 72 h of contacting with the cell culture medium.

The copolymer used alone as a dilution in the cell culture medium at a very low concentration (0.00064 mg/mL) also significantly promoted fibroblast proliferation.

It was also possible to observe that, surprisingly, when the Sepinov EMT 10 copolymer is used in combination with potassium sucrose octasulfate, an effect of synergy occurs on the fibroblast proliferation. This synergy was observed in particular when the potassium sucrose octasulfate is added to the culture medium at a concentration of 0.011 mg/mL and the Sepinov EMT 10 copolymer at a concentration of 0.00064 mg/mL.

The copolymer used according to the present invention is therefore particularly advantageous for the treatment of wounds, and in particular for promoting healing, especially when it is incorporated into a dressing.

1-12. (canceled)

13. A dressing for wound treatment, comprising
a hydrocolloid; and
an elastomeric mass comprising one or more elastomers and a copolymer of a salt of 2-methyl-2-[(1-oxo-2-propenyl)amino]-1-propanesulfonic acid and of 2-hydroxyethylpropenoate ester,
wherein the copolymer is included in an amount sufficient for treating a wound of a subject, and
wherein the amount of the copolymer is between 0.1% and 20% by weight relative to the weight of the mass into which the copolymer is incorporated.

14. The dressing as claimed in claim 13, comprising one or more active substances.

15. The dressing as claimed in claim 14, wherein said one or more active substances comprises a sulfated saccharide.

16. The dressing as claimed in claim 13, wherein said salt of 2-methyl-2-[(1-oxo-2-propenyl)amino]-1-propanesulfonic acid is a sodium salt.

17. The dressing as claimed in claim 14, wherein said one or more said active substances includes potassium sucrose octasulfate.

18. The dressing as claimed in claim 13, wherein the amount of the copolymer is between 1% and 5% by weight relative to the weight of the mass into which the copolymer is incorporated.

19. A method for promoting and/or accelerating fibroblast proliferation in vivo or ex vivo which comprises using a composition containing a copolymer of a salt of 2-methyl-2-[(1-oxo-2-propenyl)amino]-1-propanesulfonic acid and of 2-hydroxyethylpropenoate ester.

20. The method as claimed in claim 19, wherein said composition is administered to promote healing.

21. The method as claimed in claim 19, wherein said copolymer is combined with one or more active substances.

22. The method as claimed in claim 19, wherein said copolymer is combined with a sulfated saccharide.

23. The method as claimed in claim 19, wherein said salt of 2-methyl-2-[(1-oxo-2-propenyl)amino]-1-propane-sulfonic acid is a sodium salt.

24. The method as claimed in claim 21, wherein said copolymer is combined with a sulfated saccharide.

25. The method as claimed in claim 19, wherein said copolymer is combined with potassium sucrose octasulfate.

26. The dressing as claimed in claim 13, wherein the amount of the copolymer is an amount sufficient for promoting and/or accelerating fibroblast proliferation in vivo or ex vivo.

27. The dressing as claimed in claim 13, wherein the amount of the copolymer is 5% or more and less than 20% by weight relative to the weight of the mass into which the copolymer is incorporated.

28. The dressing as claimed in claim 13, wherein the elastomer is a poly(styrene-olefin-styrene) block copolymer.

29. The dressing as claimed in claim 13, wherein the hydrocolloid is sodium carboxymethylcellulose.

30. The dressing as claimed in claim 13, wherein the elastomer is a poly(styrene-olefin-styrene) block copolymer and the hydrocolloid is sodium carboxymethylcellulose.
